(19) United States
(12) Patent Application Publication
(10) Pub. No.: US 2004/0044282 A1
(43) Pub. Date: Mar. 4, 2004

(54) Medical imaging systems and methods

(76) Inventors: Lonnie Mark Mixon, Pewaukee, WI (US); Eyal Shai, Karkur (IL); Laurent Launay, Saint Remy les Chevreuse (FR); Darin R. Okerlund, Muskego, WI (US); Sarah Rose Hertel, Pewaukee, WI (US)

Correspondence Address: John S. Beulick, Armstrong Teasdale LLP, Suite 2600, One Metropolitan Sq., St. Louis, MO, 63102, US

(21) Appl. No.: 10/229,715

(22) Filed: Aug. 28, 2002

(51) Int. Cl. A61B 5/05

(52) U.S. Cl. 600/427; 600/436; 378/4; 378/901; 250/363.03; 250/363.04

(57) ABSTRACT

A method for providing diagnostic information regarding a coronary artery includes performing a Computed Tomography (CT) scan of the coronary artery to obtain structural data regarding the artery, performing a Positron Emission Tomography (PET) scan of the coronary artery to obtain functional data regarding the artery, and combining the structural data with the functional data in a single image.

### BACKGROUND OF THE INVENTION

This invention relates generally to medical imaging and, more particularly, to medical imaging of arteries and perfusion.

In at least one known method for operating a Positron Emission Tomography (PET) scanner, a PET perfusion map is obtained and the perfusion map is aligned with a generic artery overlay. However, the use of a generic artery overlay has disadvantages in that the overlay is not anatomically specific for that patient.

### BRIEF DESCRIPTION OF THE INVENTION

In one aspect, a method for providing diagnostic information regarding a coronary artery is provided. The method includes performing a Computed Tomography (CT) scan of the coronary artery to obtain structural data regarding the artery, performing a Positron Emission Tomography (PET) scan of the coronary artery to obtain functional data regarding the artery, and combining the structural data with the functional data in a single image.

In another aspect, an imaging system is provided. The imaging system includes a radiation source, a radiation detector, and a computer operationally coupled to the radiation source and the radiation detector. The computer is configured to perform a first scan of a coronary artery in a first mode to obtain structural data regarding the artery, perform a second scan of the coronary artery in a second mode different from the first mode to obtain functional data regarding the artery, and combine the structural data with the functional data in a single image.

In yet another aspect, a computer readable medium encoded with a program is provided. The program is configured to instruct a computer to perform a first scan of a coronary artery in a first mode of a medical imaging device to obtain structural data regarding the artery, and perform a second scan of the coronary artery in a second mode of the medical imaging device different from the first mode to obtain functional data regarding the artery. The program is also configured to instruct the computer to generate at least one of a wire mesh geometric model of the artery, a segmented volume of binary images of the artery, a computer program object of the artery, and a centerline trace of the artery based upon the obtained structural data. The program is also configured to instruct the computer to combine the structural data with the functional data in a single image.

In still another aspect, a computed tomography/positron emission tomography (CT/PET) medical imaging system is provided. The system includes a radiation source, a radiation detector, and a computer operationally coupled to the radiation source and the radiation detector. The computer is configured to perform a first scan of a coronary artery in a CT mode to obtain structural data regarding the artery, and perform a second scan of the coronary artery in a PET mode to obtain functional data regarding the artery including a perfusion map. The computer is also configured to combine the structural data with the functional data in a single image.

In one aspect, a computer is configured to perform a first scan of a coronary artery in a CT mode to obtain structural data regarding the artery, perform a second scan of the coronary artery in a PET mode to obtain functional data regarding the artery including a perfusion map, and generate at least one of a wire mesh geometric model of the artery, a segmented volume of binary images of the artery, a computer program object of the artery, and a centerline trace of the artery based upon the obtained structural data. The computer is also configured to combine the structural data with the functional data in a single image by three-dimensionally registering the structural data with the perfusion map.

### BRIEF DESCRIPTION OF THE DRAWINGS

FIG. 1 is a pictorial view of a CT/PET imaging system.

FIG. 2 is a block schematic diagram of the system illustrated in FIG. 2.

FIG. 3 is a screen shot of a plurality of images including portions derived from a CT scan and portions (vessels) obtained from a PET scan.

FIG. 4 is a screen shot illustrating a combination of functional data and structural data.

FIG. 5 illustrates an image of the CT rendering of the coronary artery.

### DETAILED DESCRIPTION OF THE INVENTION

In some known CT imaging system configurations, an x-ray source projects a fan-shaped beam which is collimated to lie within an X-Y plane of a Cartesian coordinate system and generally referred to as an “imaging plane”. The x-ray beam passes through an object being imaged, such as a patient. The beam, after being attenuated by the object, impinges upon an array of radiation detectors. The intensity of the attenuated radiation beam received at the detector array is dependent upon the attenuation of an x-ray beam by the object. Each detector element of the array produces a separate electrical signal that is a measurement of the beam intensity at the detector location. The intensity measurements from all the detectors are acquired separately to produce a transmission profile.

In third generation CT systems, the x-ray source and the detector array are rotated with a gantry within the imaging plane and around the object to be imaged such that the angle at which the x-ray beam intersects the object constantly changes. A group of x-ray attenuation measurements, i.e., projection data, from the detector array at one gantry angle is referred to as a “view”. A “scan” of the object comprises a set of views made at different gantry angles, or view angles, during one revolution of the x-ray source and detector.

In an axial scan, the projection data is processed to construct an image that corresponds to a two dimensional slice taken through the object. One method for reconstructing an image from a set of projection data is referred to in the art as the filtered back projection technique. This process converts the attenuation measurements from a scan into integers called “CT numbers” or “Hounsfield units”, which are used to control the brightness of a corresponding pixel on a cathode ray tube display.

To reduce the total scan time, a “helical” scan may be performed. To perform a “helical” scan, the patient is moved while the data for the prescribed number of slices is acquired. Such a system generates a single helix from a fan beam helical scan. The helix mapped out by the fan beam yields projection data from which images in each prescribed slice may be reconstructed.

Reconstruction algorithms for helical scanning typically use helical weighing algorithms that weight the collected data as a function of view angle and detector channel index. Specifically, prior to a filtered backprojection process, the data is weighted according to a helical weighing factor, which is a function of both the gantry angle and detector angle. The weighted data is then processed to generate CT numbers and to construct an image that corresponds to a two dimensional slice taken through the object.

At least some CT systems are configured to also perform Positron Emission Tomography (PET) and are referred to as CT/PET systems. Positrons are positively charged electrons (anti-electrons) which are emitted by radio nuclides that have been prepared using a cyclotron or other device. The radio nuclides most often employed in diagnostic imaging are fluorine-18 (¹⁸F), carbon-11 (¹¹C), nitrogen-13 (¹³N), and oxygen-15 (¹⁵O). Radio nuclides are employed as radioactive tracers called “radiopharmaceuticals” by incorporating them into substances such as glucose or carbon dioxide. One common use for radiopharmaceuticals is in the medical imaging field.

To use a radiopharmaceutical in imaging, the radiopharmaceutical is injected into a patient and accumulates in an organ, vessel or the like, which is to be imaged. It is known that specific radiopharmaceuticals become concentrated within certain organs or, in the case of a vessel, that specific radiopharmaceuticals will not be absorbed by a vessel wall. The process of concentrating often involves processes such as glucose metabolism, fatty acid metabolism and protein synthesis. Hereinafter, in the interest of simplifying this explanation, an organ to be imaged including a vessel will be referred to generally as an “organ of interest” and the invention will be described with respect to a hypothetical organ of interest.

After the radiopharmaceutical becomes concentrated within an organ of interest and while the radio nuclides decay, the radio nuclides emit positrons. The positrons travel a very short distance before they encounter an electron and, when the positron encounters an electron, the positron is annihilated and converted into two photons, or gamma rays. This annihilation event is characterized by two features which are pertinent to medical imaging and particularly to medical imaging using photon emission tomography (PET). First, each gamma ray has an energy of approximately 511 keV upon annihilation. Second, the two gamma rays are directed in substantially opposite directions.

In PET imaging, if the general locations of annihilations can be identified in three dimensions, a three dimensional image of an organ of interest can be reconstructed for observation. To detect annihilation locations, a PET camera is employed. An exemplary PET camera includes a plurality of detectors and a processor which, among other things, includes coincidence detection circuitry.

The coincidence circuitry identifies essentially simultaneous pulse pairs which correspond to detectors which are essentially on opposite sides of the imaging area. Thus, a simultaneous pulse pair indicates that an annihilation has occurred on a straight line between an associated pair of detectors. Over an acquisition period of a few minutes millions of annihilations are recorded, each annihilation associated with a unique detector pair. After an acquisition period, recorded annihilation data can be used via any of several different well known back projection procedures to construct the three dimensional image of the organ of interest.

As used herein, an element or step recited in the singular and preceded with the word “a” or “an” should be understood as not excluding plural said elements or steps, unless such exclusion is explicitly recited. Furthermore, references to “one embodiment” of the present invention are not intended to be interpreted as excluding the existence of additional embodiments that also incorporate the recited features.

Also as used herein, the phrase “reconstructing an image” is not intended to exclude embodiments of the present invention in which data representing an image is generated but a viewable image is not. Therefore, as used herein the term “image” broadly refers to both viewable mages and data representing a viewable image. However, many embodiments generate (or are configured to generate) at least one viewable image.

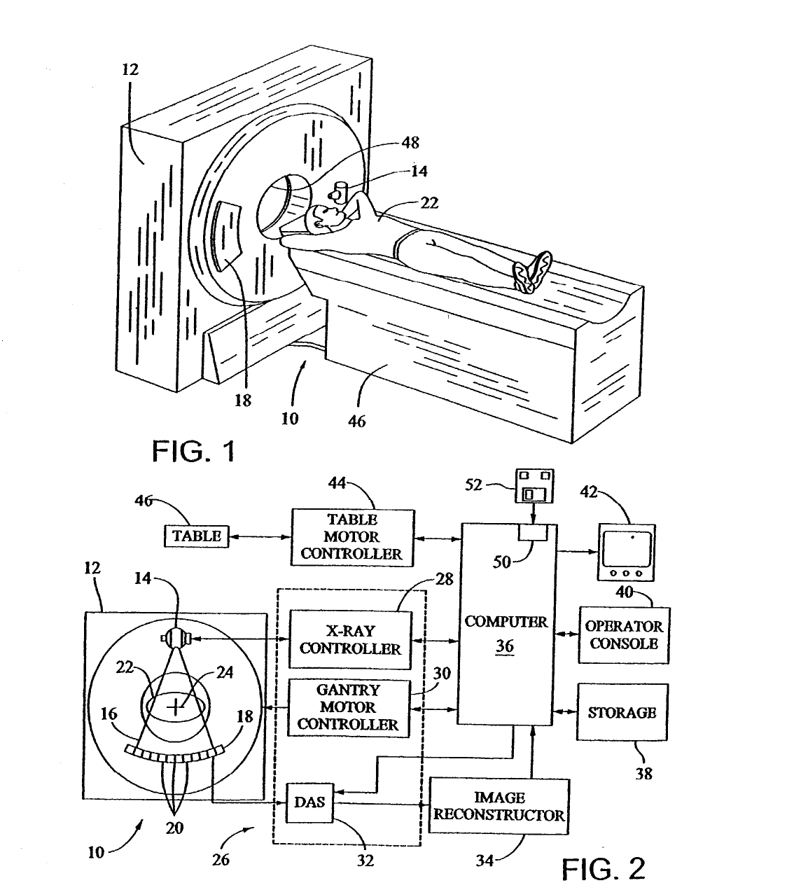

Referring to FIGS. 1 and 2, a multi-slice scanning imaging system, for example, a Computed Tomography/Positron Emission Tomography (CT/PET) imaging system 10, is shown as including a gantry 12 representative of a “third generation” CT imaging system in combination with PET circuitry. Gantry 12 has an x-ray source 14 that projects a beam of x-rays 16 toward a detector array 18 on the opposite side of gantry 12. Detector array 18 is formed by a plurality of detector rows (not shown) including a plurality of detector elements 20 which together sense the projected x-rays that pass through an object, such as a medical patient 22. Each detector element 20 produces an electrical signal that represents the intensity of an impinging x-ray beam and hence allows estimation of the attenuation of the beam as it passes through object or patient 22. During a scan to acquire x-ray projection data, gantry 12 and the components mounted thereon rotate about a center of rotation 24. FIG. 2 shows only a single row of detector elements 20 (i.e., a detector row). However, a multislice detector array 18 includes a plurality of parallel detector rows of detector elements 20 such that projection data corresponding to a plurality of quasi-parallel or parallel slices can be acquired simultaneously during a scan.

Rotation of gantry 12 and the operation of x-ray source 14 are governed by a control mechanism 26 of CT/PET system 10. Control mechanism 26 includes an x-ray controller 28 that provides power and timing signals to x-ray source 14 and a gantry motor controller 30 that controls the rotational speed and position of gantry 12. A data acquisition system (DAS) 32 in control mechanism 26 samples analog data from detector elements 20 and converts the data to digital signals for subsequent processing. An image reconstructor 34 receives sampled and digitized x-ray data from DAS 32 and performs high-speed image reconstruction. The reconstructed image is applied as an input to a computer 36 which stores the image in a storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 in gantry 12. Particularly, table 46 moves portions of patient 22 through gantry opening 48.

In one embodiment, computer 36 includes a device 50, for example, a floppy disk drive or CD-ROM drive, for reading instructions and/or data from a computer-readable medium 52, such as a floppy disk or CD-ROM. In another embodiment, computer 36 executes instructions stored in firmware (not shown). Computer 36 is programmed to perform functions described herein, and as used herein, the term computer is not limited to just those integrated circuits referred to in the art as computers, but broadly refers to computers, processors, microcontrollers, microcomputers, programmable logic controllers, application specific integrated circuits, and other programmable circuits, and these terms are used interchangeably herein. CT/PET system 10 also includes a plurality of PET cameras including a plurality of detectors. The PET detectors and detector array 18 both detect radiation and are both referred to herein as radiation detectors. In one embodiment, CT/PET system 10 is a Discovery LS CT/PET system commercially available from General Electric Medical Systems, Waukesha Wis., and configured as herein described.

CT/PET system 10 is configured to perform a Computed Tomography (CT) scan of a coronary artery to obtain structural data regarding the artery, perform a Positron Emission Tomography (PET) scan of the coronary artery to obtain functional data regarding the artery, and combine the structural data with the functional data in a single image. In one embodiment, CT/PET system 10 is also configured to generate a wire mesh geometric model of the artery based upon the obtained structural data. In another embodiment, CT/PET system 10 is also configured to generate a segmented volume of binary images of the artery based upon the obtained structural data. In yet another embodiment, CT/PET system 10 is also configured to generate a computer program object of the artery based upon the obtained structural data. In an exemplary embodiment, the computer program object is a DICOM object using a RT DICOM Object standard, where DICOM refers to Digital Imaging and Communications in Medicine and RT refers to Radiation Therapy. Alternatively, CT/PET system 10 is configured to generate a centerline trace of the artery based upon the obtained structural data. CT/PET system 10 facilitates performing a PET scan of the coronary artery to obtain a perfusion map which is three-dimensionally registered with the structural data to provide an image including anatomical data (structural data) and functional data (perfusion map).

In use, a CT scan of an artery is performed to obtain anatomical data, and a PET scan of the artery is performed to obtain functional data. The functional data and the anatomical data is combined in a single image to provide a doctor or other clinician with an image that includes functional and structural information to assist the doctor in diagnosis. This fused image can be either static or dynamic in nature while simultaneously rendering the structural and functional data.

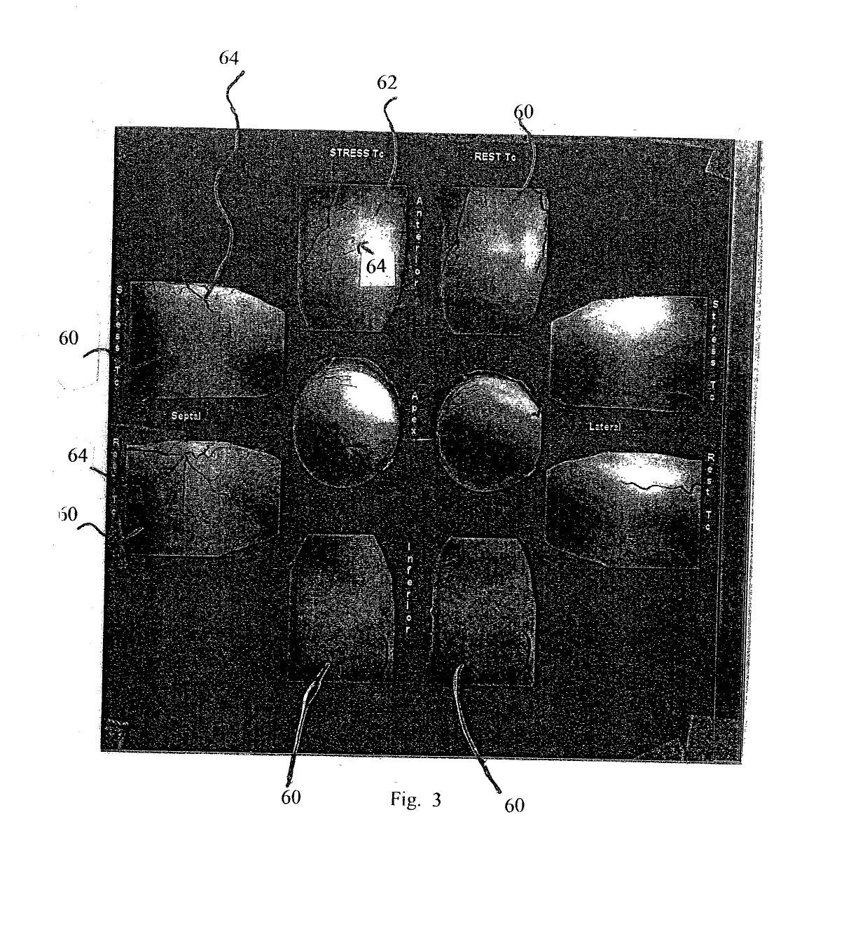

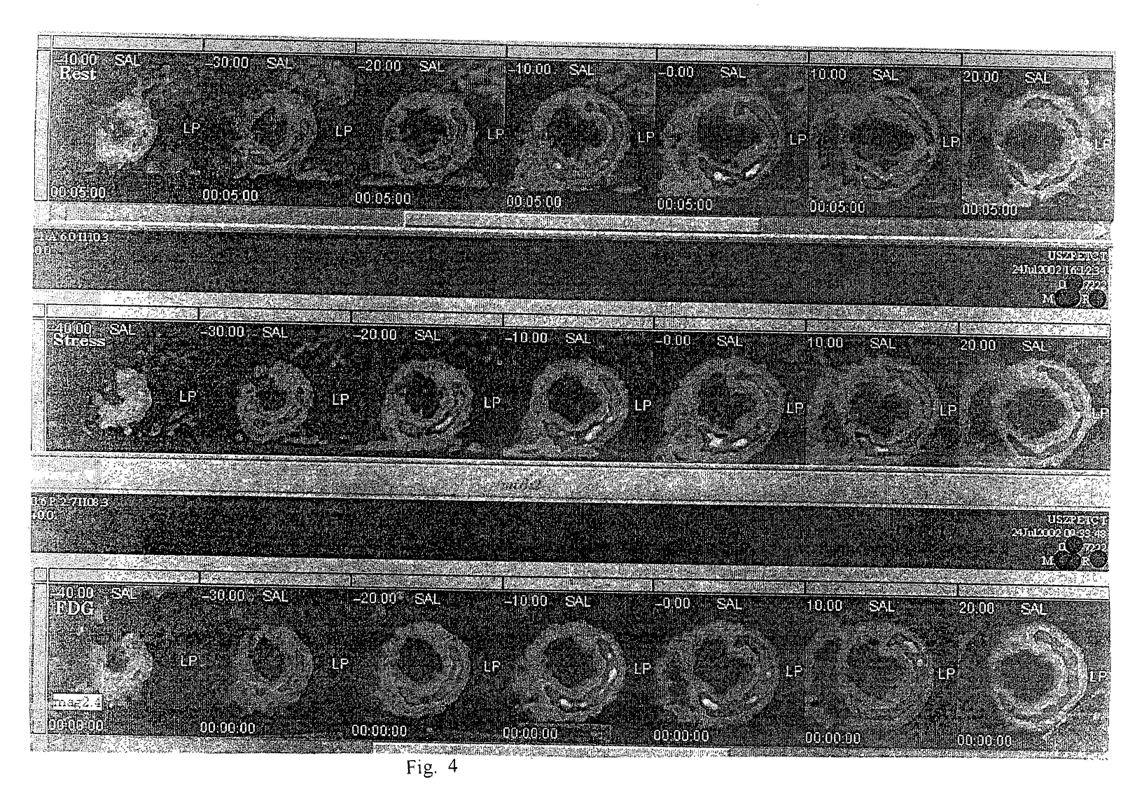

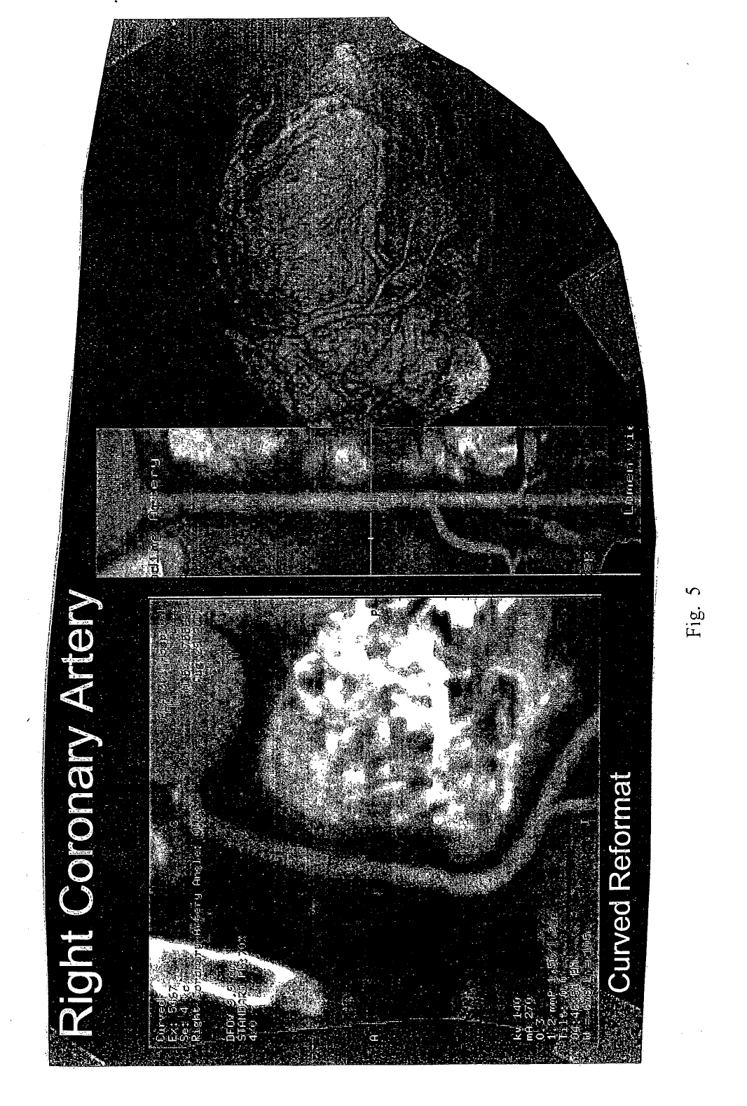

FIG. 3 is a screen shot of a plurality of images 60 including portions 62 derived from a CT scan and portions (vessels) 64 obtained from a PET scan. FIG. 4 is a screen shot illustrating a combination of functional data and structural data. FIG. 5 illustrates an image of the CT rendering of the coronary artery. Referring to FIGS. 3 and 4, rather than incorporating a generic artery overlay, FIGS. 3 and 4 illustrate images wherein a patient's particular artery structure is combined with the functional data to provide patient specific images. In one embodiment, lumen diameters are displayed. These measurements are used to determine the threshold of anatomic constrictions or lesion abnormalities, which could result in reduced perfusion to the myocardium that is supplied by that artery. The resulting perfusion defect can then be used to quantify the functional consequence of that reduced blood flow. It is contemplated that the benefits of the invention accrue to all forms of fused display modes including but not limited to 3-D and 4-D rendering; polar plots with or without normals databases, orthogonal slicing with or without triangulation; and all other modes of fused display.

While the invention has been described in terms of various specific embodiments, those skilled in the art will recognize that the invention can be practiced with modification within the spirit and scope of the claims.

What is claimed is:

1. A method for providing diagnostic information regarding a coronary artery, said method comprising:
performing a Computed Tomography (CT) scan of the coronary artery to obtain structural data regarding the artery;
performing a Positron Emission Tomography (PET) scan of the coronary artery to obtain functional data regarding the artery; and
combining the structural data with the functional data in a single image.

2. A method in accordance with claim 1 further comprising generating a wire mesh geometric model of the artery based upon the obtained structural data.

3. A method in accordance with claim 1 further comprising generating a segmented volume of binary images of the artery based upon the obtained structural data.

4. A method in accordance with claim 1 further comprising generating a computer program object of the artery based upon the obtained structural data.

5. A method in accordance with claim 1 further comprising generating a centerline trace of the artery based upon the obtained structural data.

6. A method in accordance with claim 1 wherein said performing a Positron Emission Tomography (PET) scan of the coronary artery to obtain functional data regarding the artery comprises performing a Positron Emission Tomography (PET) scan of the coronary artery to obtain a perfusion map.

7. A method in accordance with claim 6 further comprising three-dimensionally registering the structural data with the perfusion map.

8. A method in accordance with claim 1 further comprising three-dimensionally registering the structural data with the functional data.

9. A method in accordance with claim 1 further comprising displaying the image in at least one of a 3-D and a 4-D rendering mode; a polar plot with or without normals databases, and a orthogonal slicing with or without triangulation.

10. An imaging system comprising:
a radiation source;
a radiation detector; and
a computer operationally coupled to said radiation source and said radiation detector, said computer configured to:
perform a first scan of a coronary artery in a first mode to obtain structural data regarding the artery;
perform a second scan of the coronary artery in a second mode different from the first mode to obtain functional data regarding the artery; and
combine the structural data with the functional data in a single image.

11. An imaging system in accordance with claim 10 wherein said computer further configured to:
perform the first scan in a Computed Tomography (CT) mode; and
perform the second scan in a Positron Emission Tomography (PET) mode.

12. An imaging system in accordance with claim 10 wherein said computer further configured to generate a wire mesh geometric model of the artery based upon the obtained structural data.

13. An imaging system in accordance with claim 10 wherein said computer further configured to generating a segmented volume of binary images of the artery based upon the obtained structural data.

14. An imaging system in accordance with claim 10 wherein said computer further configured to generate a computer program object of the artery based upon the obtained structural data.

15. An imaging system in accordance with claim 10 wherein said computer further configured to generating a centerline trace of the artery based upon the obtained structural data.

16. An imaging system in accordance with claim 10 wherein said computer further configured to perform the second scan in a Positron Emission Tomography (PET) mode to obtain a perfusion map.

17. An imaging system in accordance with claim 16 wherein said computer further configured to three-dimensionally register the structural data with the perfusion map.

18. An imaging system in accordance with claim 10 wherein said computer further configured to three-dimensionally register the structural data with the functional data.

19. A computer readable medium encoded with a program configured to instruct a computer to:
perform a first scan of a coronary artery in a first mode of a medical imaging device to obtain structural data regarding the artery;
perform a second scan of the coronary artery in a second mode of the medical imaging device different from the first mode to obtain functional data regarding the artery;
generate at least one of a wire mesh geometric model of the artery, a segmented volume of binary images of the artery, a computer program object of the artery, and a centerline trace of the artery based upon the obtained structural data; and
combine the structural data with the functional data in a single image.

20. A computer readable medium in accordance with claim 19 wherein said program further configured to instruct the computer to:
perform the first scan in a Computed Tomography (CT) mode; and
perform the second scan in a Positron Emission Tomography (PET) mode.

21. A computer readable medium in accordance with claim 19 wherein said program further configured to instruct the computer to perform the second scan in a Positron Emission Tomography (PET) mode to obtain a perfusion map.

22. A computer readable medium in accordance with claim 21 wherein said program further configured to instruct the computer to three-dimensionally register the structural data with the perfusion map.

23. A computer readable medium in accordance with claim 19 wherein said program further configured to instruct the computer to three-dimensionally register the structural data with the functional data.

24. A Computed Tomography/Positron Emission Tomography (CT/PET) medical imaging system comprising:
a radiation source;
a radiation detector; and
a computer operationally coupled to said radiation source and said radiation detector, said computer configured to:
perform a first scan of a coronary artery in a CT mode to obtain structural data regarding the artery;
perform a second scan of the coronary artery in a PET mode to obtain functional data regarding the artery including a perfusion map; and
combine the structural data with the functional data in a single image.

25. A CT/PET medical imaging system in accordance with claim 24 wherein said computer further configured to three-dimensionally register the structural data with the perfusion map.

26. A computer configured to
perform a first scan of a coronary artery in a Computed Tomography CT mode to obtain structural data regarding the artery;
perform a second scan of the coronary artery in a Positron Emission Tomography (PET) mode to obtain functional data regarding the artery including a perfusion map;
generate at least one of a wire mesh geometric model of the artery, a segmented volume of binary images of the artery, a computer program object of the artery, and a centerline trace of the artery based upon the obtained structural data;
combine the structural data with the functional data in a single image by three-dimensionally registering the structural data with the perfusion map.

27. A computer in accordance with claim 26 further configured to displaying the image in at least one of a 3-D and a 4-D rendering mode; a polar plot with or without normals databases, and a orthogonal slicing with or without triangulation.
